(19) United States
(12) Patent Application Publication
(10) Pub. No.: US 2023/0110065 A1
(43) Pub. Date: Apr. 13, 2023

(54) COMBINATION OF ANETHOLE TRITHIONE OR ITS DERIVATIVE, 4-OH-ANETHOLE TRITHIONE, WITH DOXYCYCLINE OR MINOCYCLINE FOR THE USE THEREOF IN THE TREATMENT OF PARKINSON'S DISEASE

(71) Applicant: Olivier Petitjean, Senlis (FR)

(72) Inventor: Olivier Petitjean, Senlis (FR)

(21) Appl. No.: 17/904,069

(22) PCT Filed: Feb. 11, 2021
(86) PCT No.: PCT/FR2021/050244

§ 371 Date: Aug. 11, 2022

(30) Foreign Application Priority Data
Feb. 11, 2020 (FR) 2001355

(51) Int. Cl. A61K 31/385; A61K 31/65; A61P 25/16

(52) U.S. Cl. CPC A61K 31/385; A61K 31/65; A61P 25/16

(57) ABSTRACT

The invention relates to the field of treating Parkinson’s disease. More particularly, it relates to the use of anethole trithione on its own or in combination with an anti-synuclein tetracycline in the treatment of Parkinson’s disease.

### CROSS-REFERENCE TO RELATED APPLICATIONS

This application is a national phase entry under 35 U.S.C. § 371 of International Patent Application PCT/FR2021/050244, filed Feb. 11, 2021, designating the U.S. of America and published as International Patent Publication WO 2021/160968 A1 on Aug. 19, 2021, which claims the benefit under Article 8 of the Patent Cooperation Treaty to French Patent Application Serial No. 2001355, filed Feb. 11, 2020.

### TECHNICAL FIELD

The present disclosure relates to the field of treating Parkinson’s disease. More particularly, it relates to the use of a molecule, which inhibits the production of ROS of mitochondrial origin such as anethole trithione (ATT) or 4-hydroxy-anethole trithione (ATX), on its own or in combination with an anti-synuclein tetracycline, such as doxycycline, in the treatment of Parkinson’s disease.

### BACKGROUND

Parkinson’s disease is a chronic degenerative neurological disease affecting the central nervous system. It causes tremors, slowness in movement and muscle stiffness. This disease went untreated for a long time.

It was with the approval issued by the FDA in 1970 for L-DOPA that the treatment of Parkinson’s disease (PD) really began, a treatment, which aims to restore the dopamine deficit observed at the central level. This logic will be repeated with the placement on the market of L-DOPA, COMT and IMAO-B metabolism inhibitors, as with that of the various dopaminergic agonists. In all cases, these are strictly symptomatic supplementary treatments, which act on the motor problems and of which the effects inexorably wear off over time.

Many therapeutic targets have been identified but none of them have been clinically validated. Among these different approaches, mention can be made of rotigotine (US2007/0191470), a partial agonist of glycine (US2004/0087596), a combination of a dopaminergic agent and a nicotine receptor agonist or a nicotine receptor (EP1977746), etc.

To date, there is no etiological treatment for Parkinson’s disease.

### BRIEF SUMMARY

It is proposed that a new therapeutic approach combining the inhibition of two pathways involved in Parkinson’s disease. The proposed strategy is shown in FIG. 1. It is based on a hypothesis never put forward before, according to which the death of dopaminergic neurons is to be attributed essentially to a vicious loop, which forms between mitochondrial ROS and toxic oligomers from the a-synuclein present in the presynaptic terminals, and which is self-sustaining. Added to this primary mechanism are:
activation of the microglia, which are responsible for the release of proinflammatory factors that will contribute to mitochondrial dysfunction and thereby to the production of ROS, and
activation of MMPs, in particular, MMP3, which form a negative loop with ROS and participate in the maintenance of this destructive system. (See FIG. 2).

The present disclosure therefore proposes breaking this vicious circle by combining:
blocking the production of ROS at the mitochondrial level by administrating anethole trithione (ATT), together with
stabilizing the a-synuclein-protofibrils equilibrium in order to stop the production of toxic oligomers by means of a combined administration of doxycycline and ATT.

On the one hand, anethole trithione (ATT) has the property of blocking the production of ROS at the Iq complex of the respiratory chain (Detaille, 2019); this blockage will protect dopaminergic neurons from toxicity by MPTP (as shown in the acute model carried out in mice, WO2017/042267).

ATT can be substituted by ATX, which corresponds to the phenolic form of ATT.

On the other hand, doxycycline exhibits (i) anti-amyloid activity by binding to protofibrils, thereby blocking the production of toxic oligomers as well as the interneuronal propagation (seed effect) of these toxic oligomers (Gonzalez-Lizarraga, 2017), but also (ii) opposes the inflammatory reaction that accompanies the activation of microglia (Bortolanza, 2018) and moreover decreases the release and expression of MPPs in dopaminergic neurons (Bartolanza, 2018; Santa-Cecilia, 2019).

It should be noted that doxycycline could be replaced, in the indication for the treatment of Parkinson’s disease, by minocycline or other tetracyclines, which share several properties with doxycycline that can be used in the proposed indication.

Moreover, the inventors have shown that ATT can also inhibit the formation of toxic oligomers and is capable of playing a neuroprotective role with respect to dopaminergic neurons. This result, which is already significant on its own, is accompanied by a strong synergistic effect when ATT is combined with doxycycline.

Thus, the inventors have demonstrated that the use of ATT on its own has a significant effect on the symptoms of Parkinson’s disease at a higher dose than that at which it is usually administered, namely at a maximum daily dose of 75 mg (see SURFALEM® dosage); in the treatment of Parkinson’s, the dose considered is at least 300 mg per day.

Thus, the present disclosure relates to the use of ATT or ATX, on its own or in combination with an anti-synuclein tetracycline, for the treatment of Parkinson’s disease.

### Advantages of the Present Disclosure

The present disclosure constitutes the first etiological therapeutic approach to Parkinson’s disease. It allows a reversal of symptoms maintained over the long term (at least 3 years, with the current follow-up) and therefore brings a real benefit to the patient.

The observations collected on a clinical case are confirmed by analyses carried out in vitro on models, which are representative of the different physiological aspects of the disease. The experimental results presented below attest to a direct effect of ATX on two major components of Parkinson’s disease, namely the activation of microglia and the aggregation of a-synuclein, which they both inhibit. These results alone make ATX (or ATT) a candidate of interest in the treatment of Parkinson’s disease. In addition, it is shown that the combination of ATX with an a-synuclein inhibitor, in this case doxycycline, has a synergistic effect on the reduction of inflammation. This combination also acts synergistically on the neuroprotection of dopaminergic neurons. It is moreover very interesting for avoiding the occurrence of failure mechanisms. The combination also makes it possible to inhibit the “seed effect” (or propagation effect) and therefore to prevent the progression of the disease.

Beneficial effects of a treatment combining ATT and a tetracycline are expected in all Parkinson’s patients, at least by inducing stabilization of the patient’s condition. However, it is foreseeable that the administration of this treatment in patients at an early stage, as soon as the first symptoms appear, or even as a preventive measure in patients likely to develop the disease, will allow a very significant (possibly total) regression of symptoms, and that the patient will be able to maintain this state over the long term.

Making this innovative therapeutic approach available to all Parkinson’s patients will be facilitated by the fact that it is based on the use of molecules available on the market and of which the harmlessness has been established for a long time. This aspect is very favorable to its adoption by doctors and patients.

### BRIEF DESCRIPTION OF THE DRAWINGS

FIG. 1: Graphical representation of the therapeutic approach, which is the subject of the present disclosure, combining the inhibition of two pathways involved in Parkinson’s disease; these pathways create a vicious loop, which combines mitochondrial ROS and toxic oligomers from the a-synuclein present in the presynaptic terminals, and which is self-sustaining. According to ESK El Sayed, 2018 and RM Meade, 2019.

FIG. 2: Schematic view of the cell death mechanism observed at the neuronal level in Parkinson’s disease; according to Bortolanza, 2018; El Sayed, 2018; Guo, 2018; Liu, 2018; Mead, 2019; Santa Cecilia, 2019. (Abbreviations: ETC - Electron Transport Chain; MMP - Matrix metalloproteinase; mPTP - mitochondria permeability transition pore; mtDNA -mitochondrial DNA; ROS - Reactive Oxygen Species).

FIG. 3: Impact of treatments with ATX, DOX or ATX + DOX on microglia in culture activated with LPS - A - Anti-inflammatory effect of ATX on microglia in culture activated by LPS. Measurement by means of an ELISA test of the release of TNF-a in cultures of microglia exposed to LPS (10 ng/mL) for 24 hrs in the presence or absence of ATX (1-30 µM) or DEX (2.5 µM). The data represent the mean ± SEM values expressed as % of the measured values in the sole presence of the inflammogen (n=3-7 wells/condition), ****p<0.0001 vs. control; ^{##}p<0.01, ^{####}p<0.0001 vs. LPS. B - Anti-inflammatory effect of DOX on microglia in culture activated by LPS. Measurement by means of an ELISA test of the release of TNF-a in cultures of microglia exposed to LPS (10 ng/mL) for 24 hrs in the presence or absence of DOX (10-100 µM) or DEX (2.5 µM). The data represent the mean ± SEM values expressed as % of the measured values in the sole presence of the inflammogen (n=3-9 wells/condition). ****p<0.0001 vs. control; ####p<0.0001 vs. LPS. C - Anti-inflammatory effect of a treatment combining ATX + DOX on microglia in culture activated by LPS. Measurement by means of an ELISA test of the release of TNF-a in cultures of microglia exposed to LPS (10 ng/mL) for 24 hrs in the presence or absence of ATX (10 µM), DOX (50, 100 µM), the two combined treatments (ATX + DOX) or DEX (2.5 µM). The data represent the mean ± SEM values expressed as % of the measured values in the sole presence of the inflammogen (n=5-13 wells/condition). ****p<0.0001 vs. control; ###p<0.001, ####p<0.0001 vs. LPS; &&p<0.01 vs. ATX or DOX separately.

FIG. 4: Impact of treatments with ATX, DOX or ATX + DOX on microglia in culture activated with PAM3CSK4 - A - Anti-inflammatory effect of ATX on microglia in culture activated by PAM3CSK4. Measurement by means of an ELISA test of the release of TNF-a in cultures of microglia exposed to PAM3CSK4 (1 µg/mL) for 24 hrs in the presence or absence of ATX (5-30 µM) or DEX (2.5 µM). The data represent the mean ± SEM values expressed as % of the measured values in the sole presence of the inflammogen (n=4-12 wells/condition), ****p<0.0001 vs. control; #p<0.05, ####p<0.0001 vs. PAM3CSK4. B - Anti-inflammatory effect of DOX on microglia in culture activated by PAM3CSK4. Measurement by means of an ELISA test of the release of TNF-a in cultures of microglia exposed to PAM3CSK4 (1 µg/mL) for 24 hrs in the presence or absence of DOX (10-100 µM) or DEX (2.5 µM). The data represent the mean ± SEM values expressed as % of the measured values in the sole presence of the inflammogen (n=4-8 wells/condition), ****p<0.0001 vs. control; #p<0.05, ###p<0.001, ####p<0.0001 vs. PAM3CSK4. C - Anti-inflammatory effect of a treatment combining ATX + DOX on microglia in culture activated by PAM3CSK4. Measurement by means of an ELISA test of the release of TNF-a in cultures of microglia exposed to PAM3CSK4 (1 µg/mL) for 24 hrs in the presence or absence of ATX (5 µM), DOX (50, 100 µM), and the two combined treatments or DEX (2.5 µM). The data represent the mean ± SEM values expressed as % of the measured values in the sole presence of the inflammogen (n=4-12 wells/condition). ****p<0.0001 vs. control; #p<0.05, ##p<0.01, ####p<0.0001 vs. LPS; &p<0.05 vs. ATX or DOX separately.

FIG. 5: Inhibitory effects of ATX, DOX or ATX + DOX on amyloid aggregation of a-synuclein - A - Effects of DOX and DMF on amyloid aggregation of a-synuclein. Fluorescence intensity emitted by ThT under conditions where monomeric a-synuclein (αSm) is shaken (600 rpm, 37° C., 96 h) in the presence or absence of DOX (100 µM) or DMF (1%). Comparison with αSm, which is not shaken. The experimental data represent the mean values ± SEM in % of the values obtained under the conditions where the monomers are shaken on their own without treatment (n=6-8 measurements/condition), ****p<0.0001 vs. Control; ####p<0.0001 vs. shaken αSm. B - Effect of ATX, optionally combined with DOX, on the amyloid aggregation of a-synuclein. Fluorescence intensity emitted by ThT under conditions where the monomeric a-synuclein is shaken (600 rpm, 37° C., for 96 h) in the presence or absence of DOX (10 µM) or ATX (1, 10 or 100 µM). The experimental data represent the mean ± SEM values in % of the values obtained under the conditions where the monomers are shaken on their own without treatment (n=6-8 measurements/condition), **p<0.01, ****p<0.0001 vs. shaken aS.

FIG. 6: Transmission electron microscopy demonstrating the anti-aggregation effect of the ATX compound. Images of a-synuclein aggregates produced in the presence of ATX (10 µM; aS:ATX), DOX (10 µM; aS:DOX) or the two treatments combined (aS:DOX:ATX). Scale bars: 1 µm (4,000X) and 500 nm (10,000X).

FIG. 7: Neuroprotective potential of ATX in relation to dopaminergic neurons subjected to low-noise, spontaneous oxidative stress. Illustration describing the neuroprotective effects of ATX (3 µM), DOX (D-3447, Sigma Adrich; 10 µM) or apotransferrin (APO; T-1428, Sigma Adrich; 150 µg/mL) in 7-day-old midbrain cultures.

FIG. 8: Cooperative neuroprotective effects of ATX and DOX on dopaminergic neurons in culture subjected to low-noise, spontaneous chronic oxidative stress. Survival of dopaminergic neurons in midbrain cultures optionally treated for 7 days with apotransferrin (APO; 150 µg/mL), ATX (0.75 µM; 3 µM), DOX (7.5 µM; 10 µM) or an ATX (0.75 µM) + DOX (7.5 µM) combination. The experimental data represent the mean ± SEM values in % of the values obtained in the presence of apotransferrin (n=4-13 wells/condition), ***p<0.001 vs. controls; ^{###} p<0.001 vs. the same concentration of ATX or DOX applied separately.

### DETAILED DESCRIPTION

A first object of the present disclosure relates to the use of a molecule, which inhibits the production of ROS of mitochondrial origin such as anethole trithione (ATT) or its derivative 4-OH-anethole trithione (ATX) for treating Parkinson’s disease, in which the daily dose is between 300 mg and 1200 mg.

ATT and ATX are two specific inhibitors of ROS of mitochondrial origin.

ATX corresponds to the phenolic form of ATT as it is metabolized by the liver, both in humans and in animals. This 4-OH-anethole trithione form has been previously described (Li et al., J Pharm Biomed Anal, 2008, 47: 612-617). Since the structure of ATT is preserved during this metabolization, there is every reason to think that the antiROS activity carried by ATT is found in ATX, especially since after oral administration, which is currently the form marketed, the main part of the circulating product found is ATX (Yu, 2011). In addition, ATX carries a para-phenol group, which allows the formation of esters.

In a particular embodiment, ATX is used in its esterified form, for example, in the ester form of: phosphate, ethylidene phosphate, sulfate, hemisuccinate, acetate, propionate, isobutyrate, hexanoate, pivalate, ethoxycarbonate, nicotinate, or even ester of amino acids such as glycine, diethylglycine or valine ester, and the list is not exhaustive.

The proposed dose is markedly higher than the doses proposed in the prior art for treating diseases, which cause peripheral damage. Indeed, the dose of 300 mg to 1200 mg per day is considered necessary to allow the active molecule to reach the neurons after crossing the blood-brain barrier. This dose can be adjusted according to the stage of the disease and/or the patient, for example, to 400 mg, 500 mg, 600 mg, 700 mg, 800 mg, 900 mg, 1000 mg or 1200 mg per day.

It is preferred to administer this dose in 2, 3 or 4 doses distributed at regular intervals over the day in order to ensure as much as possible a constant level of ATT or ATX in dopaminergic neurons, which are target cells in this pathology. Preferably, the administration is divided into 3 doses. For a better quality of life for the patient, it can be taken in 2 parts.

The doses for ATX are the same as those proposed for ATT insofar as ATX carries most of the activity of ATT since ATT metabolizes almost immediately and completely into ATX via O-demethylation, to the point that almost no ATT can be found in the patient’s urine over a 24-hour period (He J. et al. J Pharm Biomed Anal. 2011, 54: 551-556; Li TM. et al. Anal Chim Acta 2007, 594: 274-278; Li WY. et al. J Pharm Biomed Anal. 2008, 47: 612-617).

The use of ATT or ATX monotherapy can be considered as primary preventive treatment for the onset of symptoms or to alleviate or eliminate the first symptoms and slow down or stop the progression thereof. Indeed, ATT has an inhibitory effect both on the inhibition of inflammation and on the formation of toxic aggregates in dopaminergic neurons, which makes it possible to prevent the spread of the disease linked to the seed effect. Thus, in a preferred embodiment of the present disclosure, ATT is administered to a patient exhibiting the first symptoms of the disease (early stage) or to a patient likely to develop Parkinson’s disease.

The ATT treatment is intended, in particular, for a patient at an early stage of the disease or for an asymptomatic patient.

An “asymptomatic patient” is understood to mean a patient diagnosed as likely to develop Parkinson’s disease, but who does not present symptoms.

ATT or ATX can also be administered to a patient treated with L-DOPA. In this embodiment, it is expected that the dyskinetic symptoms experienced by patients treated for a long time with L-DOPA will improve. In addition, administering an ATT-tetracycline combination makes it possible to control the symptoms with the consequence that the L-DOPA dose can be reduced, so that the side effects of dyskinesia disappear.

A second object of the present disclosure relates to the use of a combination of (i) a molecule, which inhibits the production of ROS of mitochondrial origin such as ATT or its derivative ATX and (ii) an anti-synuclein tetracycline, for treating Parkinson’s disease.

“Anti-synuclein tetracyclines” are understood to mean doxycycline, minocycline, glycylcycline, eravacycline, omadacycline, tetracycline, oxytetracycline, chlortetracycline or one of their derivatives.

“Doxycycline derivatives” are understood to mean 9-amino-doxycycline and 9-aminomethyl-doxycycline, and their respective acylated derivatives, as well as 9-amino-4-dedimethylamino-doxycycline and 9-aminomethyl-4-dedimethylamino-doxycycline, and their respective acylated derivatives.

“Minocycline derivatives” are understood to mean 9-amino-minocycline and 9-aminomethyl-minocycline, and their respective acylated derivatives.

In a particular embodiment of this use, the daily dose of ATT or its derivative ATX is between 300 mg and 1200 mg and the daily dose of doxycycline or minocycline or one of their derivatives is between 30 mg and 200 mg.

Doses may vary depending on the stage of the disease and/or the patient. The dose of doxycycline or minocycline may, for example, be between 30 mg and 60 mg, or between 60 mg and 90 mg or between 90 mg and 120 mg or even between 120 mg and 200 mg per day. This daily dose is divided into 2, 3 or 4 doses, preferably into 2 or 3 doses.

In a preferred embodiment, the combination comprises ATT and doxycycline.

The combination of ATT or ATX with doxycycline or minocycline can be proposed in a preventive or curative approach to the occurrence of a mechanism in which the established treatments fail, possibly in addition to such treatments.

In a preferred embodiment of the present disclosure, the ATT and tetracycline combination is administered to a patient exhibiting the first symptoms of the disease (early stage) or to a patient likely to develop Parkinson’s disease. In these patients, the expected effects are more significant, namely the disappearance of symptoms or prevention of the appearance thereof and stabilization of the disease in the long term.

The ATT + tetracycline combination is intended, in particular, for a patient at an early stage of the disease or for an asymptomatic patient.

This combination of molecules can be administered to a patient undergoing treatment with L-DOPA. These two treatments can be considered complementary: the ATT/ATX-tetracycline combination allows two major mechanisms of action in Parkinson’s disease to be inhibited, while L-DOPA compensates for the loss of function of dopaminergic neurons. Thus, the two treatments can act in a complementary manner, on the one hand on the cause of the loss of dopaminergic neurons and on the other hand on the dopamine level to alleviate the symptoms present. Combining these two treatments can be completely advantageous, especially if one of the targeted therapeutic pathways fails.

A third object of the present disclosure relates to a pharmaceutical composition comprising a molecule, which inhibits the production of ROS of mitochondrial origin such as ATT or ATX, an anti-synuclein tetracycline and carriers.

Such a composition can be formulated so as to allow 2 or 3 medicinal doses per day. By way of example for 3 daily doses, such a composition may comprise between 100 mg and 400 mg of ATT or ATX and between 10 mg and 65 mg of doxycycline or minocycline or of one of their derivatives. For 2 daily doses, such a composition may comprise between 150 mg and 600 mg of ATT or ATX and between 15 mg and 98 mg of doxycycline or minocycline or of one of their derivatives.

In a preferred embodiment, the pharmaceutical composition comprises ATT and doxycycline.

A fourth object of the present disclosure relates to the use of a pharmaceutical composition comprising (i) a molecule, which inhibits the production of ROS of mitochondrial origin such as ATT, (ii) an anti-synuclein tetracycline and (iii) carriers for treating Parkinson’s disease.

This composition can be administered to a patient treated with L-DOPA, in particular, to a patient exhibiting symptoms despite taking L-DOPA medication.

A fifth object of the present disclosure relates to a chimera, between (i) ATX and (ii) an anti-synuclein tetracycline derivative such as doxycycline or minocycline.

“Chimera,” also referred to as a condensation product, is understood to mean the molecule, which is formed after a condensation reaction between 4-OH-anethole trithione (ATX) and one of the derivatives in position 9 of doxycycline or minocycline.

In a preferred embodiment, the chimera comprises the conjugation of ATX with a doxycycline derivative.

The present disclosure will be better understood on reading the examples that follow, which are provided by way of illustration and should in no case be considered as limiting the scope of the present disclosure.

### EXAMPLES

### Example 1: Description of a Clinical Case

This therapeutic solution was tested on a 69-year-old man with Parkinson’s disease. A history of events over 3 years (current follow-up) is presented below:

### Stage 1: Effect of ATT on the Symptoms of Parkinson’s Disease

Symptoms: At the very beginning of 2018, the first manifestations of Parkinson’s appeared: tremors in the left hand, slowing of walking pace, chaotic descent of stairs, tilting of the torso forward accompanied by back pain in the form of muscle contractures, as well as the beginnings of various obvious non-motor disorders (withdrawal, indifference, difficult decision-making, loss of joy in life, depression, etc.), constipation, hyposmia. Initially, ATT was tested as monotherapy.

Treatment: The problem then arose of deciding which dose to use. The patient was already taking ATT to treat a problem of benign prostatic hypertrophy (cf. patent WO2019/O73173) and used it with the greatest satisfaction at a dose of 3 cp to 25 mg, given in the morning and evening, but the dose turned out to be insufficient for treating Parkinson’s disease. A blood-brain barrier crossing problem was suspected and the decision was taken to increase this dose by 4 times by dividing it into 4 daily doses, i.e., 4 x 6 tablets/day (equivalent to 600 mg/day), so as to reduce concentration fluctuations as much as possible (the plasma half-life of ATT in humans is approximately 3 hrs).

Result: Quite spectacularly, three days later, all the clinical symptoms disappeared, except for the tremors, hyposmia and constipation.

These initial results constitute the first proof of concept of the beneficial effect of ATT on Parkinson’s disease. In a correlated manner, they confirm the hypothesis that ROS, in particular, mitochondrial ROS, are involved in Parkinson’s disease.

### Stage 2: Effect of the Combination of ATT and Doxycycline

Symptoms: In April-May 2019, unusual fatigue manifested when the patient performed strenuous tasks.

In July 2019, the appearance of balance problems in a context of profound fatigue justified the introduction of SINEMET®; L-DOPA solved the problem of loss of balance but the hand tremors only slightly improved and fatigue persisted as did the difficulty experienced by the patient in speeding up his walking pace, and constipation also persisted. Micrographia appeared.

Treatment: Faced with worsening fatigue, the decision was taken, on Dec. 19, 2019, to add doxycycline at a dose of 50 mg every 6 hrs, which dose was intended to ensure stable concentrations in the LCR of approximately 0.9 µM/L (Dotevall, 1989; Karlsson, 1996), i.e., around 3 times what Gonzalez-Lizzaraga (2017) recommends.

Results: Five days later, the patient’s fatigue problems considerably regressed, and he managed to walk at a faster pace; on D10, the impression of fatigue disappeared and the patient’s walking pace practically returned to normal, while his hand tremors were almost no longer perceptible. After one month of treatment, the patient rarely experienced back pain and the micrographia disappeared. At the same time, it was possible to reduce the doses of SINEMET® from the first weeks by 20-25%, and today (February 21) by 50-60%. Finally, since Jan. 15, 2020, the dosage has been adjusted to 3 administrations per day [3 times 8 tablets of 25 mg of ATT + 3 times 50 mg of doxycycline] without a loss of treatment benefits.

It is important to add that no significant side effects have been noted during these two years of treatment, apart from intestinal meteorism linked to taking ATT. The patient’s condition is stable.

In summary:
13 satisfactory months on ATT (150 mg, 4 times a day)
Followed by 7 months of failure with the onset of new symptoms (always 150 mg of ATT, 4 times a day)
13 satisfactory months on ATT + DOX (600 mg of ATT + 150 mg of DOX per day).

Conclusion: The treatment failure with ATT and then the recovery observed after resorting to doxycycline constitute a first validation of the reality of these two targets, ROS and toxic oligomers of a-synuclein, which cooperate in a process leading to the death of central dopaminergic neurons. This cooperation therefore implies that the therapy chosen, in order to be fully effective and sustainable over time, is directed toward each of these two targets at the same time.

### Example 2: Effect of the ATX and Doxycycline Combination on the Activation of Microglia

### 1- Materials and Methods

Protocols using animals: The mice used for this study were housed and handled according to Directive 2010/63/EU of the European Parliament and of the Council of Sep. 22, 2010. The experimental protocols were approved by the Ethics Committee for Animal Experimentation, Charles Darwin no. 5.

Purification of microglia: The cultures were produced from a cell suspension obtained by mechanical dissociation of the brains of newborn mice, C57BL/6J (Janvier LABS), according to a protocol described by Sepulveda-Diaz and colleagues (Glia, 2016) and modified subsequently by dos-Santos-Pereira and colleagues (Glia, 2020). The protocol used follows the directives of the Council of the European Union (2010/63/EU). It was approved by the Ethics Committee for Animal Experimentation, Charles Darwin no. 5. Corning culture flasks of 75 cm² precoated with a polycation, polyethyleneimine (PEI; 1 mg/mL) diluted in a borate buffer pH 8.3, were used so as to obtain spontaneous isolation of the microglial cells after 15-20 days of culture, this isolation taking place at the expense of other cell populations, in particular, astrocytes. In order to maintain the flask cultures, Dulbecco’s modified Eagle’s medium (DMEM) supplemented with 10% fetal bovine serum (FBS, Biowest LLC) and 1% penicillin/streptomycin cocktail was used (see experimental details described in the publication of Sepulveda-Diaz and colleagues). After the microglia purification step, the microglia adhering in the culture flasks were trypsinized to produce subcultures in 48-well multi-well Nunc plates. These cultures were maintained in the presence of DMEM, supplemented with 20 mM HEPES and 1% FBS.

Culture processing paradigms: The stimulation of microglia was obtained by a 24-hour treatment with two reference inflammogens, LPS (10 ng/mL), a Toll-like receptor (TLR)4 agonist (Dos-Santos-Pereira et al, 2020) or PAM3CSK4 (1 µg/mL), a TLR2 agonist (Acuna et al, Cells, 2019). 4-hydroxy-anethole trithione (ATX), doxycycline (DOX) and dexamethasone (DEX; 2.5 µM), used as reference anti-inflammatory, were added 4 hrs before triggering the activation of microglial cells by the inflammogens. The stock solution of ATX was at 25 mM in DMSO and that of DOX was at 10 mM in distilled water. The mechanisms of the anti-inflammatory effects of DOX on microglial cells were previously reported by Santa-Cecilia and colleagues (Neurotox Res, 2016).

Assessment of TNF-a levels: The culture medium of the microglia, recovered 24 hrs after the stimulation by the inflammogens was initiated, was then frozen at -20° C. until the analysis. TNF-a was detected with an ELISA kit from ThermoFisher (BMS607-3TWO), following the supplier’s instructions (dos-Santos-Pereira et al, Glia, 2020). The absorbance of each sample was measured at 450 nm with a SpectraMax i3X.

Statistical analysis: The experimental data were analyzed with the GraphPad Prism 8.3.1 software by applying a One-Way ANOVA test, followed by a post-hoc Student Newman Keuls test.

### 2- Results

The effect of an ATX and doxycycline combination on inflammation in a culture of microglia activated in vitro was compared with the effect of each of the molecules individually. The activation of microglia was tested in two different experimental models, the activation being mimicked either by LPS or by PAM3CSK4.

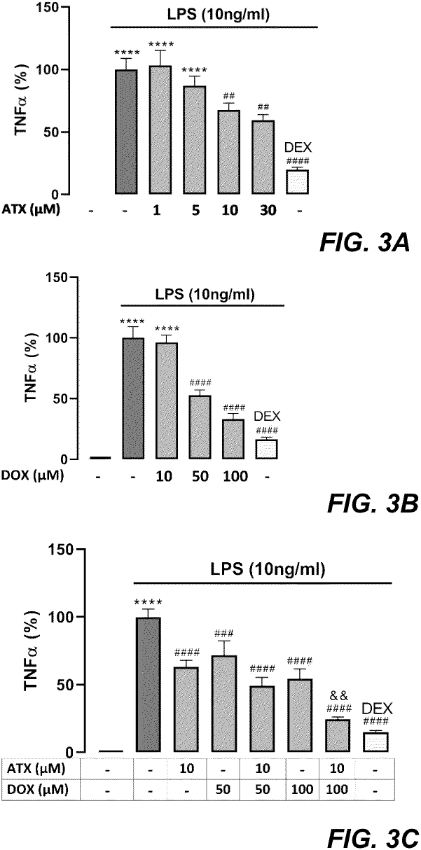

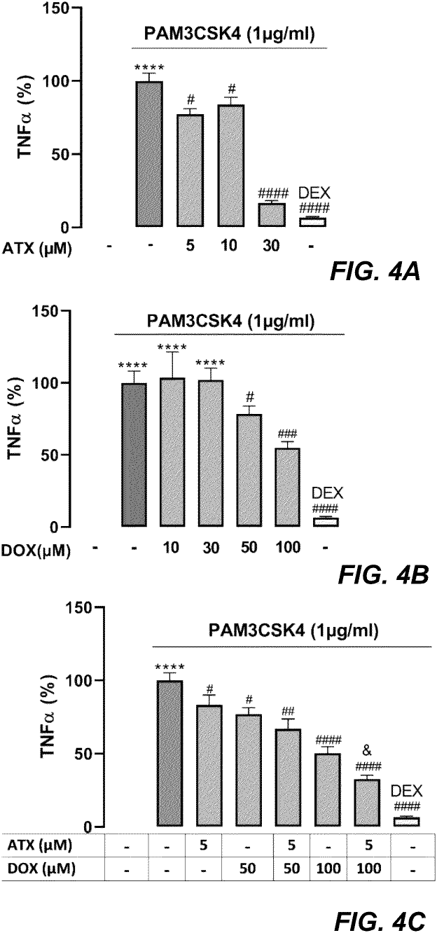

The results obtained on microglia activated by LPS are shown in FIG. 3. The results obtained on microglia activated by PAM3CSK4 are shown in FIG. 4.

It was observed that ATX on its own is capable of inhibiting the release of TNF-a, an inflammation marker (FIGS. 3A and 4A). Doxycycline (DOX) on its own also induced a decrease in inflammation (FIGS. 3B and 4B). Very interestingly, the ATX + DOX combination improved the inhibition of the release of TNF-a compared with that obtained with each of the molecules tested separately (FIGS. 3C and 4C).

### 3- Discussion of Results

In a cellular model allowing the reproduction of the neuroinflammatory component of the disease (Sepulveda-Diaz et al, 2016), it is shown here that ATX exerted robust anti-inflammatory effects (at 10 or 30 µM, depending on the activation paradigm) on reference inflammogens, lipopolysaccharide (LPS) or PAM3CSK4, respectively, agonists of the TLR-4 and TLR-2 receptors. In the same experimental paradigm, DOX exerted anti-inflammatory effects between 50 and 100 µM. By testing different combinations of ATX and DOX, it was possible to show that some of them are at the origin of more robust anti-inflammatory effects than those produced by each compound, tested separately, without leading, stricto sensu, to synergistic effects as they are classically defined.

However, the desired synergistic effect seems difficult to demonstrate here insofar as the responses observed on the basis of the concentrations used are not linear, whether it is ATX or DOX, the effects lessening as the concentrations are increased (e.g., in the LPS model: 50 µM of DOX inhibited the release of TNFa by 50% when 100 µM inhibited it by approximately 70%; in the same way, 30 µM of ATX did not lower the release of TNFa observed after 10 µM by a factor of 3, but rather only by 15 to 20%); however, in this context, still in the LPS model, it was observed that the mere fact of adding to each of the 2 doxycycline arms, DOX 50 and DOX 100, the same concentration of 10 µM of ATX increased the gains observed with doxycycline used on its own when its concentration was increased from 50 to 100 µM (cf. FIG. 3B). The same thing was found with the PAM3CSK4 model (cf. FIG. 4). This phenomenon is the expression of the synergistic potential of the combination, a judgment, which is reinforced by the fact that, in the LPS model, 10 µM of ATX + 100 µM of DOX do almost as well as the reference dexamethasone.

### Example 3: Effect of ATX on the Aggregation of α-Synuclein

### 1- Materials and Methods

Preparation of a-synuclein: Human recombinant a-synuclein was produced from a strain of Escherichia coli carrying a plasmid pT7-7 containing the coding sequence of the protein (Kaylor et al, J Mol Biol, 2005; Gonzalez-Lizarraga et al, Sci Rep, 2020). After a purification step, the purity of the protein was evaluated by polyacrylamide gel electrophoresis under denaturing conditions (SDS-PAGE). The stock solution of a-synuclein was prepared in a buffer containing 20 mM of HEPES and 150 mM of NaCl at pH 7.4. Before initiating the aggregation protocol, the protein was centrifuged at 12000 g for 30 minutes in order to eliminate the micro-aggregates. The protein concentration was measured at 280 nm with a Nanodrop 8000 spectrophotometer (Gonzalez-Lizarraga et al, Sci Rep, 2020).

Aggregation of a-synuclein ex vitro: The aggregation protocol was adapted from previous work (Kaylor et al, J Mol Biol, 2005; Gonzalez-Lizarraga et al, Sci Rep, 2020). Briefly, the aggregation of a-synuclein was obtained by orbital shaking (600 rpm; Thermomixer Comfort; Eppendorf), at 37° C. for 96 h, of the recombinant protein (70 µM) solubilized in 20 mM of HEPES and 150 mM of NaCl, pH 7.4. Dimethylformamide (DMF) was used to dilute ATX in this experimental protocol. The formation of aggregated structures was monitored by means of the Thioflavin-T (ThT) test (LeVine et al, Meth Enzymol, 1999).

### 2- Results

The ability of the ATX + DOX combination to prevent the aggregation of a-synuclein was evaluated in comparison with the effect of each of the molecules tested separately.

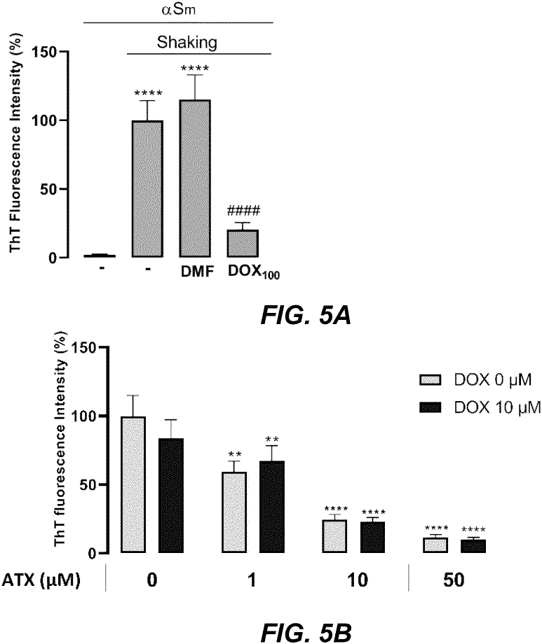

The results obtained on an ex vitro model are shown in FIG. 5.

It was observed that DOX is capable of inhibiting this aggregation, as confirmed in FIG. 5A.

Unexpectedly, ATX also has this property. Moreover, the inhibition caused by ATX is effective, even at a low concentration (FIG. 5B).

The ATX + DOX combination did not allow the inhibition advantage to be improved in this model. However, combining two molecules acting through separate metabolic pathways is particularly promising for preventing failure mechanisms.

Having demonstrated that ATX has an inhibitory effect both on the inflammation of microglia and on the aggregation of a-synuclein is a very encouraging result for its use in the treatment of Parkinson’s disease, especially in combination with an a-synuclein inhibitor such as doxycycline.

### 3- Discussion of Results

This experimental work made it possible to demonstrate that the ATX molecule is capable of effectively and concentration-dependently inhibiting the aggregation of human recombinant a-synuclein (aS) in an ex vitro test in which the amyloid aggregation of the monomeric form of the protein was obtained by orbital shaking (Gonzalez-Lizarraga et al, 2017). The inhibitory effects of ATX, observable between 1 and 50 µM, were however not amplified by DOX, which was used at 10 µM, that is to say a concentration devoid of its own anti-aggregation effect, an indirect way of recalling that the mechanisms of action of the two molecules are fundamentally different: DOX stabilizes the soluble monomeric-tetrameric form of a-synuclein while ATT blocks the overproduction of mitochondrial ROS responsible for redirecting the future of a-synuclein toward its aggregated “toxic oligomeric” form.

### Example 4: Anti-Aggregation Effect of α-Synuclein of the ATX Compound Visualized by Transmission Electron Microscopy

### 1- Materials and Methods

Transmission electron microscopy: The a-synuclein samples, shaken in the optional presence of the treatments of interest, were characterized by transmission electron microscopy. Aliquots of 50 µl of each solution were used for this analysis. The sample preparation protocol is described in detail by Gonzalez-Lizarraga and colleagues (2020). Images were obtained with a transmission electron microscope, Philips EM 301.

### 2- Results

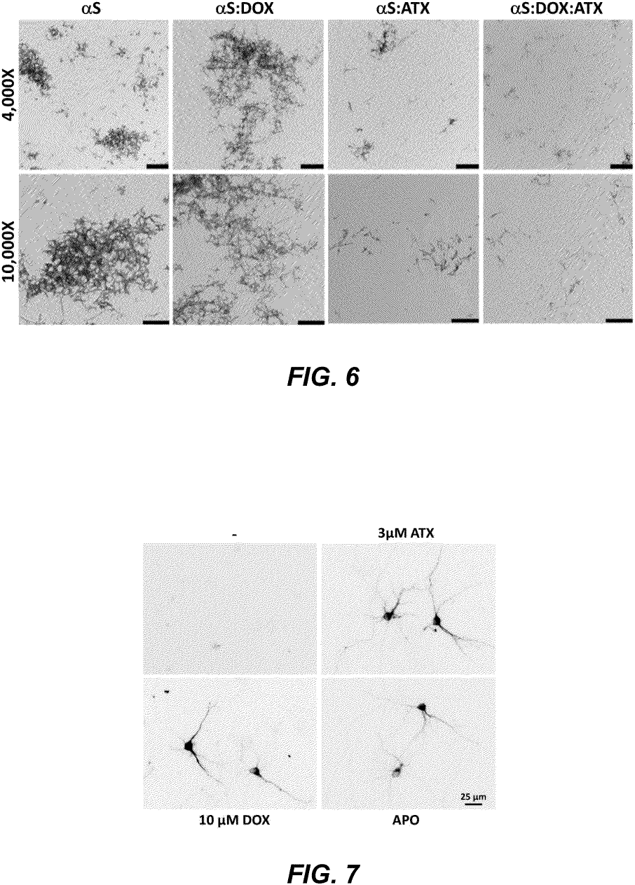

The results are shown in FIG. 6. These images confirm that ATX (10 µM) effectively reduces the amyloid aggregation of a-synuclein, whereas DOX has no effect at this same concentration. An effect comparable to that produced by 10 µM of ATX was obtained with 100 µM of DOX (Gonzalez-Lizarraga et al, 2020). A cooperative effect of the two molecules could not be demonstrated in this experiment.

### Example 5: Neuroprotective Effect of ATT on Dopaminergic Neurons in Culture Subjected to Low-Noise, Spontaneous Oxidative Stress, ATT Being on its Own or in Combination with Doxycycline

### 1- Materials and Methods

Midbrain cultures and immunodetection of tyrosine hydroxylase (TH): Mouse midbrain cultures were produced based on a protocol initially described by Troadec and colleagues (2001) and subsequently taken up by Rousseau and colleagues (2013). The cultures were generated from Swiss mouse embryos (Janvier LABS, Le Genest-St Isles) taken from the pregnant mother on day 13.5 of gestation. The cells dissociated from the midbrain were cultured in 48-well plates containing 500 µl of DMEM/HamF12 medium, supplemented with 10% fetal bovine serum (Biowest LLC, Les Ulis, France). Approximately 80-100 10³ cells were added initially to each culture well. After one hour, the initial medium was replaced with the DMEM/HamF12 medium supplemented with 20 µg/mL of insulin. The culture medium (350 µl) and the treatments were renewed on days 1 and 3 in vitro. The cultures were maintained for a total of 7 days before binding and analysis. The presence of trace Fe (II) sulfate (1.5 µM) in the DMEM/HamF12 medium was sufficient to trigger low-noise, spontaneous oxidative stress causing the progressive loss of dopaminergic neurons in the midbrain cultures (Rousseau et al, 2013). The reference cultures were treated with 150 µg/mL of apotransferrin (APO; T-1428, Sigma Aldrich). The immunodetection of tyrosine hydroxylase was carried out with a mouse anti-tyrosine hydroxylase antibody (Immunostar #22941) of which the presence was revealed with a 2^{sd} anti-mouse antibody, labeled Alexa-Fluor 555. TH⁺ neurons were counted according to a protocol described previously (Rousseau et al, 2013). The counts are expressed as % of the values obtained in the control cultures treated with apotransferrin.

Statistical analysis: The experimental data correspond to mean ± SEM values. The number of individual values (n) for each experimental point results from at least two independent experiments. The data were analyzed with the GraphPad Prism 8.3.1 software or Sigmaplot 12.5 software using One-Way ANOVA, followed by a post-hoc Student Newman Keuls test.

### 2- Results

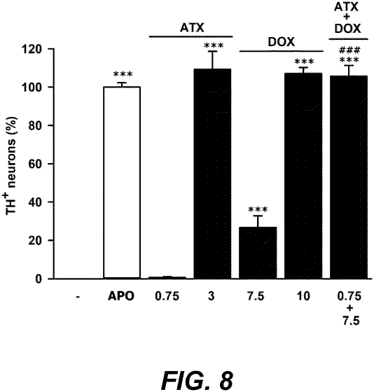

The results are shown in FIGS. 7 and 8.

Treatment with apotransferrin represents the reference protective treatment. It should be noted that in the absence of treatment the neurons immunolabeled with TH virtually disappeared from the cultures. Fluorescence images are presented in an inverted format.

FIG. 7 shows the protective effect of ATT, which even appears to be superior to that of DOX and apotransferrin.

FIG. 8 compares the effect of 2 concentrations of ATT, 2 concentrations of DOX, and the result of the combination thereof. For ATT, the 2 concentrations tested were 0.75 and 3 µM; the concentration of 3 µM exhibited a protective effect comparable to the reference treatment, in this case apotransferrin, but almost no effect at 0.75 µM (protection equivalent to 0.6% of that of APO). Regarding DOX, for which 2 concentrations were also tested, 7.5 and 10 µM, the maximum effect was observed at a concentration of 10 µM while at 7.5 µM the protective effect of DOX only reached 27% of that of apotransferrin. Very interestingly, the combination of ATT at 0.75 µM with DOX at 7.5 µM provided protection equivalent to that of APO, which clearly demonstrates a synergistic effect of the two molecules.

### 3- Discussion of Results

The neuroprotective potential of ATX is demonstrated here in a cellular model of dopaminergic neuronal degeneration in which DOX is also protective (see FIGS. 7 and 8). The evaluation of different combinations of DOX and ATX, using concentrations of each compound, having little or no effect, is in progress, but the first series of experimental data presented clearly demonstrate cooperative effects, in particular, for the ATX (0.75 µM) + DOX (7.5 µM) combination (FIG. 8).

### Conclusion

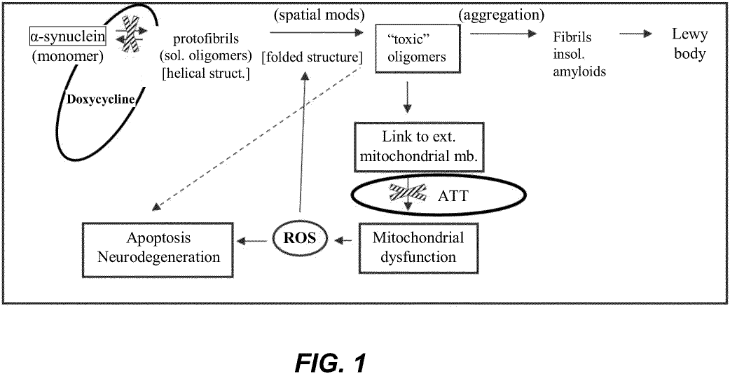

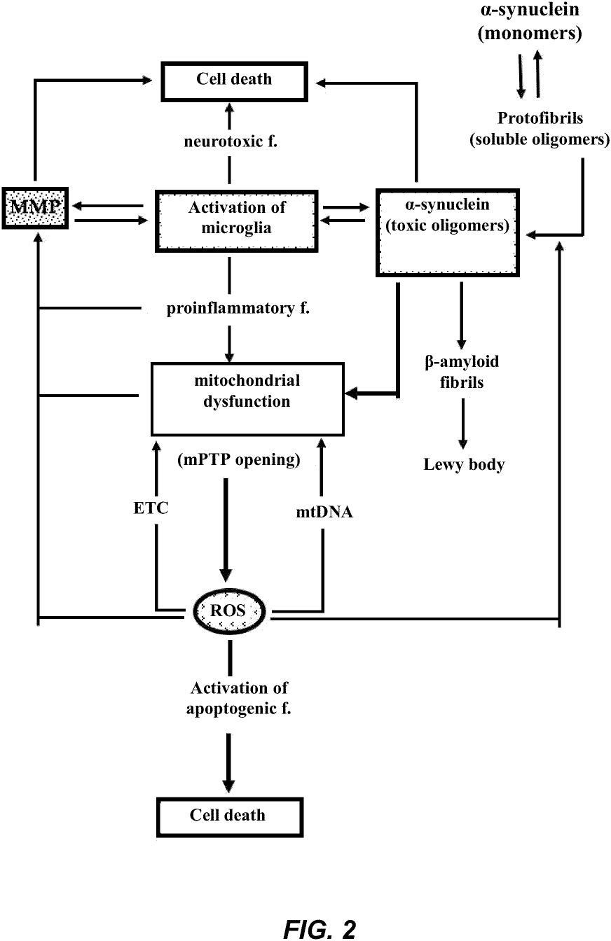

It is important to emphasize that it is thanks to different and complementary mechanisms of action that this ATT + DOX combination manages to break this vicious circle, which is self-sustaining and ultimately leads to neuronal degeneration, and where the following sequences are successively linked under the impact of the disease:
soluble a-synuclein, which self-aggregates and forms toxic oligomers,
these toxic oligomers, which will attach to the external membrane of the mitochondria and cause the dysfunction thereof, which induces ipso facto electronic leakage and hyperproduction of ROS,
finally, closing the loop, the overproduction of ROS, which redirects the production of a-synuclein from its native soluble form to the toxic aggregated form,
and, simultaneously, the system spreads step by step, from cell to cell via the seeding effect. See FIG. 1.

It is by breaking this negative loop, which allows the disease to prosper that the ATT-DOX combination protects the patient from the risk of treatment failure to which the choice of monotherapy exposes him, as illustrated very well by the clinical case reported in the patent; note that the patient in question is 14 months into treatment with the ATT-DOX combination and that for the moment everything is going well for him.

Finally, very interestingly, in this work, the anti-aggregation effects of ATX were confirmed by transmission electron microscopy.

### Perspectives - Continuation of the Experimental Project

The remainder of the project will consist in validating that the ATT-DOX treatment can inhibit dyskinesia induced by L-DOPA. The first step will be to establish a model of dyskinesias induced by L-DOPA to test the anti-dyskinetic effect of ATT, administered on its own or concomitantly with DOX. DOX will be used as an anti-dyskinetic reference molecule (Bortolanza et al, 2020). It will be a question of determining if ATX, like DOX, exerts anti-dyskinetic effects and if DOX/ATX combinations improve the behavior of the animals in a cooperative manner. The rodent model of dyskinesias that will be used will consist in inducing a unilateral lesion of the substantia nigra by stereotaxic injection of 6-hydroxydopamine at a “medial forebrain bundle” level, so as to generate a dopaminergic neuronal loss of > 90% in the substantia nigra, causing near total dopamine depletion in the ipsilateral dorsal striatum. The extent of the neuronal loss will be confirmed by an apomorphine rotation test, and the injured rats will be evenly distributed in the different experimental groups, according to the response of the animals to this test. Two weeks after the lesion (day 14), the injured rats will be rendered dyskinetic by daily treatments with L-DOPA and benserazide HCl (a peripheral DOPA decarboxylase inhibitor). These treatments will be carried out over a period of two weeks. Ten days after the initiation of the L-DOPA treatments (day 24), the compounds of interest (DOX, ATT) will be administered, separately or in combination, and the abnormal movements or AIMS (Abnormal Involuntary Movements) in these animals will be evaluated according to protocols codified and described in detail in the literature (Dos-Santos Pereira et al, 2021; Bortolanza et al, 2020). Finally, the animals will be tested to determine if the compounds of interest do not interfere with the expected motor effects of L-DOPA. To this end, the horizontal behavior, stereotypies and rearing will be measured under L-DOPA, in the optional presence of the treatments of interest, using an automated video monitoring system.

1. (canceled)

2. (canceled)

3. (canceled)

4. A composition comprising a combination of a molecule that inhibits the production of reactive oxygen species of mitochondrial origin chosen from anethole trithione or its derivative 4-OH-anethole trithione, and an anti-synuclein tetracycline.

5. The composition of claim 4, wherein the tetracycline is chosen from doxycycline, minocycline or one of their derivatives.

6. The composition of claim 5, wherein the combination is in the form of a single dosage of anethole trithione or its derivative 4-OH-anethole trithione of between 300 mg and 1200 mg and a dosage of doxycycline or minocycline or their derivatives of between 30 mg and 200 mg.

7. The composition of claim 4, wherein the molecule comprises anethole trithione and the anti-synuclein tetracycline comprises doxycycline.

8. (canceled)

9. (canceled)

10. A pharmaceutical composition, comprising a molecule that inhibits production of reactive oxygen species of mitochondrial origin selected from among anethole trithione or its derivative 4-OH-anethole trithione, and an anti-synuclein tetracycline and carriers.

11. The pharmaceutical composition of claim 10, further comprising between 100 mg and 400 mg of anethole trithione or its derivative 4-OH-anethole trithione, and between 10 mg and 65 mg of a tetracycline chosen from doxycycline, minocycline, or one of their derivatives.

12. The pharmaceutical composition of claim 10, wherein the pharmaceutical composition comprises anethole trithione and doxycycline.

13. (canceled)

14. (canceled)

15. A chimera between 4-hydroxy-anethole trithione and one of the C9 derivatives of doxycycline or minocycline.

16. A method of treating Parkinson’s disease in a human patient, comprising administration of a therapeutically effective amount of a composition comprising a combination of a molecule that inhibits the production of reactive oxygen species of mitochondrial origin and an anti-synuclein tetracycline.

17. The method of claim 16, wherein the molecule is chosen from anethole trithione or its derivative 4-OH-anethole trithione.

18. The method of claim 16, wherein the patient is undergoing treatment with L-DOPA.

19. The method of claim 16, further comprising administering the composition to the patient at a daily dosage of from 300 mg to 1200 mg.

20. The method of claim 16, further comprising administering the composition to the patient at a daily dosage of between 100 mg and 400 mg of anethole trithione or its derivative 4-OH-anethole trithione, and at a dosage between 10 mg and 65 mg of a tetracycline chosen from doxycycline, minocycline, or one of their derivatives.
